(19) United States
(12) Patent Application Publication
(10) Pub. No.: US 2005/0266081 A1
(43) Pub. Date: Dec. 1, 2005

(54) Antimicrobial silver hydrogels

(76) Inventor: Wallace J. Rogozinski, Azusa, CA (US)

Correspondence Address: Min, Hsieh & Hack, LLP;c/o PortfoliolP, P.O. Box 52050, Minneapolis, MN, 55402, US

(21) Appl. No.: 10/853,152

(22) Filed: May 26, 2004

(51) Int. Cl. A61K 33/38; A61K 9/14

(52) U.S. Cl. 424/484; 424/618

(57) ABSTRACT

An antimicrobial hydrogel composition contains at least one antimicrobial silver salt; at least one viscosity-enhancing agent chosen from natural clay and synthetic clay; and at least one electrolyte. Methods of making the composition, methods of disinfecting, and methods of treating are also disclosed.

### FIELD

The present invention relates to compositions for disinfecting substrates, including tissue, and methods of disinfection. The inventive compositions comprise at least one antimicrobial silver salt, at least one viscosity-enhancing agent, and at least one electrolyte.

### INTRODUCTION

Silver was among one of the first metals known to man that exhibited anti-infective properties. Ancient antidotal accounts of the use of silver to maintain the potability of water are scattered throughout history. While many of these early descriptions of silver's powers are attributed to myth or to the black art of the alchemist, silver, nevertheless was recognized to possess legitimate therapeutic value.

During the second half of the nineteenth century, bacteriology became a true and respected science. Several drugs and treatments based upon silver were developed during this time when an understanding of the basis of infectious disease and the anti-infective properties of chemical and biological agents became known.

The treatment of ophthalmia neonatorum with a 1% silver nitrate solution applied to each eye (Crede's prophylaxis, 1884) was regarded as a medical milestone. B. C. Crede, a surgeon, also began the use of silver in wound antisepsis about 1897 and pioneered the use of silver in skin infections.

Development of silver colloids for anti-infective applications progressed in the twentieth century and led to the introduction of silver sulfadiazine in 1968. Since that time, silver sulfadiazine has become the standard of care for burns.

With the advent of antibiotics, most topical treatments containing silver preparations fell into disuse. However, the liberal use of antibiotics brought about a serious crisis in the management of infectious diseases in the form of antibiotic resistant microorganisms. The emergence of bacterial resistance to a battery of formally effective agents coupled with an inadequate spectrum of action exposed the Achilles heel of antibiotics. Consequently, the use of silver in the treatment of wounds and burns has undergone a renewed interest.

Bioburden reduction and the prevention of infection has become a goal in advanced wound care treatment protocols of modern medicine. Quantitatively, it has been shown that open wounds can maintain a bioburden of approximately 10⁵ microorganisms without the clinical manifestations of infection. Bioburden of greater than 10⁵ represent a significant challenge for local tissue defenses in the wound environment. A clinical wound infection usually results when 10⁶ or more microorganisms per gram of tissue.

Certain silver compounds in low concentrations have been acknowledged as effective broad-spectrum antimicrobials without the potential for genetic adaptation of pathogenic microorganisms and the development of resistance. However, some silver preparations exhibit adverse and toxic properties when used in the administration of burns and wounds. Ricketts et al., “Mechanism of prophylaxis by silver compounds against of burns,” Br. Med. J., (1970), pp. 444-446, determined that a 30% inhibition of skin cell respiration, caused by the application of 1 to 10 mg/ml of silver, was a probable factor in the interference of wound healing.

Silver nitrate, the most widely used of silver compounds, may be problematic because it can cause methemoglobinema through the reduction of nitrates to nitrite by bacteria. Moreover, silver nitrate in the eye can cause cauterization of the cornea if concentrations exceed 1% and exposure exceeds one minute.

Currently, there are several wound dressings sold commercially that have incorporated silver for its antimicrobial properties. For example, U.S. Pat. No. 5,753,251, discloses a wound dressing that is manufactured using a sputter coating technique, where silver is deposited onto substrates such as plastic film. U.S. Pat. No. 2,934,066, discloses a vapor deposition process whereby certain woven fibers are rendered antimicrobial. While such medical devices are useful in varying degrees, they are limited in that they require moisture for activation of silver ions from the substrate. Because the release of silver ions is completely dependent on the amount of moisture available, some silver impregnated dressings may be ineffective due to a lack of a disproportionate amount of moisture present to allow the silver ions to migrate to the intended site. Additionally, some of these dressings exhibit instability in light and may be photo-reduced to a less active state.

Wounds vary in size and shape, and are often present with a condition called undermining or tunneling, wherein there is tissue destruction underneath the visible periphery of the wound. It is, therefore, unlikely that a silver ion released from an impregnated dressing can actually reach the undermined wound areas in sufficient quantity to provide the antimicrobial dose to either prevent or treat infection.

Silver creams, such as silver sulfadiazine, are slow to release silver ions from its oily matrix and offer no means of absorbing tissue fluid which retards the delivery of silver ions. Additionally, silver sulfadiazine has a potential for cross-sensitivity with other sulfonamides that are used to treat infectious disease processes further restricting use.

The present invention may provide a method for treating skin sites or wounds that harbor infection-causing microorganisms. The antimicrobial silver hydrogel composition may interfere with the microorganisms' reproductive mechanisms. This has the effect of inhibiting their multiplication and/or causing their death by the quick release of therapeutic quantities of silver ions from an ionically bonded hydrogel structure. The antimicrobial hydrogel composition may therefore prevent and/or treat infectious disease without suppressing host defenses and/or exhibiting cytotoxic properties. The compositions of the present invention may also absorb wound exudates and other serosanguineous fluids that support the growth of pathogenic microorganisms, as well as cause the maceration of the skin around the wound margin that can retard healing.

The present invention may also reduce the numbers of microorganisms that constitute a preinfection state (wound bioburden) to host manageable levels so that a natural sequence of wound healing can occur.

The present invention may also provide a method for maintaining the peripheral area around endogenous devices, such as intravenous and urinary indwelling catheters and/or any medical device that breaches the skin, vascular system or urinary tract free of infectious microorganisms.

### SUMMARY

One embodiment of the invention is an antimicrobial hydrogel composition comprising at least one antimicrobial silver salt; at least one viscosity-enhancing agent chosen from natural clay and synthetic clay; and at least one electrolyte.

An additional embodiment of the invention is a method for topically disinfecting a substrate, which comprises applying to the substrate an effective amount of an antimicrobial composition comprising at least one antimicrobial silver salt; at least one viscosity-enhancing agent chosen from natural clay and synthetic clay; and at least one electrolyte.

A further embodiment of the invention is a method of treating a topical infection, which comprise applying to a patient in need thereof an effective amount of an antimicrobial composition comprising at least one antimicrobial silver salt; at least one viscosity-enhancing agent chosen from natural clay and synthetic clay; and at least one electrolyte to the infected area and/or the surrounding infected area.

A further embodiment of the invention is a method of treating a heavily contaminated or infected wound, which comprises applying to a patient in need thereof an effective amount of a composition comprising at least one antimicrobial silver salt; at least one viscosity-enhancing agent chosen from natural clay and synthetic clay; and at least one electrolyte to the contaminated or infected wound and/or the surrounding contaminated or infected area.

Still a further embodiment of the invention is a method of disinfecting an intact skin site prior to a surgical or invasive procedure, which comprises applying to a patient in need thereof an effective amount of a composition comprising at least one antimicrobial silver salt; at least one viscosity-enhancing agent chosen from natural clay and synthetic clay; and at least one electrolyte.

Another embodiment of the invention is a method of making an antimicrobial hydrogel composition comprising: (a) combining at least one viscosity-enhancing agent chosen from natural clay and synthetic clay with water; (b) combining at least one antimicrobial silver salt with water; (c) combining the silver salt solution from (b) with the viscosity-enhancing solution from (a) to form a thickened solution; and (d) combining at least one electrolyte with the thickened solution to form the antimicrobial hydrogel composition.

It is to be understood that both the foregoing general description and the following description of various embodiments are exemplary and explanatory only and are not restrictive.

### DESCRIPTION OF VARIOUS EMBODIMENTS

The compositions of the present invention are safe and effective, broad-spectrum topical antimicrobial compositions and may be in the form a thixotropic, non-cytotoxic hydrogel. In various embodiments, the antimicrobial hydrogel composition may comprise at least one antimicrobial silver salt, at least one viscosity-enhancing agent chosen from natural clay and synthetic clay, and at least one electrolyte. By varying the concentration of the at least one viscosity-enhancing agent, the hydrogel composition may have consistencies that range from a heavy liquid to a thick, slightly cloudy gel.

The at least one antimicrobial silver salt may provide at least the anti-infective properties of the composition. The at least one antimicrobial silver salt may be a silver lactate which conforms to the empirical formula (C₃H₅AgO₃.H₂O). Additional non-limiting examples of the at least one antimicrobial silver salt include, silver nitrate, silver acetate, silver citrate, silver picrate, and silver chloride. Silver lactate may be used for medical applications to avoid the potential for the adverse side effect, methemoglobinemia, associated with the most frequently used silver salt, silver nitrate.

The at least one antimicrobial silver salt may be present in the composition in an amount ranging from about 0.01% to about 10%, for example from about 0.01% to about 5.0% by weight relative to the total weight of the composition.

In an embodiment, the composition of the invention may comprise at least one viscosity-enhancing agent. At least one viscosity-enhancing agent refers to any agent that, when applied in various concentrations in an aqueous medium, results in the formation of stable hydrogels that exhibit thixotropic properties. The at least one viscosity-enhancing agent may be present in the composition in an amount ranging from about 0.1 to about 10% by weight with respect to the total weight of the composition. The at least one viscosity-enhancing agent may be chosen from natural clay and synthetic clay. In an embodiment, the hydrogel viscosity may be achieved by the use of an entirely synthetic mineral which is akin to the natural clay mineral hectorite in structure and composition. Unlike natural clay, a synthetic mineral is typically free of impurities yet is equal in structure to natural hectorite. One such synthetic mineral is listed in the American Chemical Society's Chemical Abstracts Service (CAS) under the name sodium lithium magnesium silicate (Registration No. 53320-86-8) and in the Cosmetic, Toiletries and Fragrance Association (CTFA) dictionary as sodium magnesium silicate. This synthetic mineral is sold commercially under the trade name LAPONITE®, a registered trademark of Southern Clay Products, Inc., Gonzales, Tex. Other non-limiting examples of the at least one viscosity-enhancing agent include magnesium aluminum silicates, smectite clays, and an amorphous clay mineral, such as allophone; two-layer type crystalline clay minerals, such as equidimensional crystal, kaolinite, and nacarite; elongate crystals, such as halloysites; three-layer type crystalline clay minerals, such as sodium montmorillonite, calcium montmorillonite, sauconite, vermiculite, nontronite, saponite, hectorite, and bentonite; chain structure crystalline clay minerals, such as attapulgite, sepiolite, and palygorskite; and mixtures thereof.

The two-layer type crystalline clay minerals are sheet structures composed of units of one layer of silica and one layer of alumina octahedrons. The three-layer type crystalline clay minerals are sheet structures composed of two layers of silica tetrahedrons and one central dioctahedral or trioctahedral layer. The chain structure crystalline clay minerals are hornblende-like chains of silica tetrahedrons linked together by octahedral groups of oxygen and hydroxyls containing aluminum and magnesium atoms.

In an embodiment, the at least one viscosity-enhancing agent may conform to the empirical formula Na_{0.7+}((Si₈Mg_{5.5}Li_{0.3})O₂₀(OH₄))^{-0.7}. The at least one viscosity-enhancing agent may serve as the gel matrix once ionic bonding has been completed.

Without being limited to any particular theory, it is believed that the swelling properties of the natural and synthetic clay minerals permit colloidal particles to form upon hydration. These colloidal particles may exhibit repulsive electrical surface charges, which may then be able to maintain a uniform suspension in solution. With the addition of an ionic compound, such as for example sodium chloride, potassium chloride, silver lactate, or any other silver salt that will ionize in solution, to the colloidal suspension, the repulsive particle charges may be reduced significantly, allowing the formation of a viscous, aqueous gel with rheologocial characteristics that may be typical of the clay mineral used. The formed gel may demonstrate at least one property such as the flow properties and the rheological behavior classically termed thixotropic, wherein a semi-solid gel may be induced by shaking or stirring, to become a sol (a thin liquid) and revert once again to a semi-solid gel upon standing.

In an embodiment, at least one organic modifier may be combined with the at least one viscosity-enhancing agent in order to realize the best properties of both. The at least one viscosity-enhancing agent and the at least one organic modifier may be used in a combination, such as an approximate ratio of about 4 parts at least one viscosity-enhancing agent to about 1 part at least one organic modifier. The at least one organic modifier may generally be cellulosic in nature, and may typically be used in the art to form thixotropic gels. Non-limiting examples of the at least one organic modifier include hydroxypropyl methyl cellulose, guar hydroxypropyl trimonium chloride, carbomer, xanthan gum, polyethylene glycol (PEG) block polymers, and polyvinylpyrrolidone.

The composition of the invention may also comprise at least one electrolyte. In various embodiments, the at least one electrolyte may be sodium chloride USP, hydrochloric acid NF, or citric acid USP. Other compounds, including alkali metal and alkali earth metal salts that dissociate into electrolytes such as the salts of potassium, magnesium, and calcium can also be used to initiate ionic bonding in the formation of thixotropic gels. Alternative electrolytes may produce gels with properties equivalent to those utilizing sodium chloride USP. Without being limited to any particular theory, it is believed that the at least one electrolyte frees up the ions in the at least one antimicrobial silver salt and may reduce the overall pH of the composition.

The at least one electrolyte may be present in the composition in an amount ranging from about 0.01% to about 10% by weight with respect to the total weight of the composition.

The antimicrobial silver hydrogel composition has a wide variety of uses, including the effective treatment of topical bacterial and fungal infections, the treatment of heavily contaminated or infected wounds, and the preparation of an intact skin site prior to a surgical or invasive procedure.

A topical infection may be understood by those of ordinary skill in the art to refer generally to a minor infection, bacterial and/or fungal in nature, which may be typically superficial and localized.

A heavily contaminated wound may be understood by those of ordinary skill in the art to mean a wound that is heavily contaminated by micro-organisms, but not clinically infected. Such wounds may be often characterized by a prolonged period of inflammation, as well as a delay in wound healing or repair. Heavily infected wounds may be understood by those of ordinary skill in the art to mean wounds with a bioburden greater than 10⁵ microorganisms per gram of tissue.

The rheological characteristics of thixotrophy, in which the apparent viscosity decreases as the system is disturbed by stirring or shaking and then reverses during periods of dormancy, may be useful in the administration and use of the invention described herein. The ability to apply a product to the skin with the use of simple delivery devices such as pump sprayers and squeeze tubes eliminates the characteristic disadvantages of dispensing thin liquids and thick gels, where thin liquids cannot be contained at the treatment site and permanently thick gels cannot be easily dispensed. The rheological phase shift from gel to sol to gel provides product administration latitude.

### EXAMPLES

The following examples are illustrative and are non-limiting to the present teachings.

### Manufacturing Methods for Antimicrobial Hydrogel

### Example 1

From about 0.1% to about 10% by weight LAPONITE®, a registered trademark of Southern Clay Products, Inc., Gonzales, Tex., depending upon the desired final viscosity, is slowly added to USP purified water under vigorous agitation and mixed until the LAPONITE® is fully hydrated and a uniform, viscous liquid forms and appears clear.

From about 0.01% to about 10% by weight silver lactate powder, manufactured by Spectrum Chemical Mfg. Corp. (Gardena, Calif.), is dispersed in an aliquot of USP purified water under vigorous agitation and mixed until completely dissolved. The silver lactate solution is then slowly added to the LAPONITE® solution and mixed vigorously until the viscosity of the mixture increases perceptibly.

From about 0.01% to about 10% by weight sodium chloride USP is very slowly added to the LAPONITE® and the silver lactate mixture under continuous and vigorous agitation. The viscosity of the mixture increases immediately and in the final composition, forms a slightly hazy, thick, semi-solid hydrogel.

### Example 2

From about 0.1% to about 10% by weight LAPONITE®, a registered trademark of Southern Clay Products, Inc., Gonzales, Tex., depending upon the desired final viscosity, is slowly added to USP purified water under vigorous agitation and mixed until the LAPONITE® is fully hydrated and a uniform, viscous liquid forms and appears clear.

From about 0.01% to about 10% by weight silver lactate powder, manufactured by Spectrum Chemical Mfg. Corp. (Gardena, Calif.), is dispersed in an aliquot of USP purified water under vigorous agitation and mixed until completely dissolved. The silver lactate solution is then slowly added to the LAPONITE® solution and mixed vigorously until the viscosity of the mixture increases perceptibly.

From about 0.01% to about 10% by weight citric acid USP is very slowly added to the LAPONITE® and the silver lactate mixture under continuous and vigorous agitation. The viscosity of the mixture increases immediately and in the final composition, forms a slightly hazy, thick, semi-solid hydrogel.

### Example 3

From about 0.1% to about 10% by weight LAPONITE®, a registered trademark of Southern Clay Products, Inc., Gonzales, Tex., depending upon the desired final viscosity, is slowly added to USP purified water under vigorous agitation and mixed until the LAPONITE® is fully hydrated and a uniform, viscous liquid forms and appears clear.

From about 0.01% to about 10% by weight silver lactate powder, manufactured by Spectrum Chemical Mfg. Corp. (Gardena, Calif.), is dispersed in an aliquot of USP purified water under vigorous agitation and mixed until completely dissolved. The silver lactate solution is then slowly added to the LAPONITE® solution and mixed vigorously until the viscosity of the mixture increases perceptibly.

From about 0.01% to about 10% by weight hydrochloric acid NF is very slowly added to the LAPONITE® and the silver lactate mixture under continuous and vigorous agitation. The viscosity of the mixture increases immediately and in the final composition, forms a slightly hazy, thick, semi-solid hydrogel.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “less than 10” includes any and all subranges between (and including) the minimum value of zero and the maximum value of 10, that is, any and all subranges having a minimum value of equal to or greater than zero and a maximum value of equal to or less than 10, e.g., 1 to 5.

It is noted that, as used in this specification and the appended claims, the singular forms “a,” “an,” and “the,” include plural referents unless expressly and unequivocally limited to one referent. Thus, for example, reference to “an electrolyte” includes two or more different electrolytes. As used herein, the term “include” and its grammatical variants are intended to be non-limiting, such that recitation of items in a list is not to the exclusion of other like items that can be substituted or added to the listed items.

It will be apparent to those skilled in the art that various modifications and variations can be made to various embodiments described herein without departing from the spirit or scope of the present teachings. Thus, it is intended that the various embodiments described herein cover other modifications and variations within the scope of the appended claims and their equivalents.

1. An antimicrobial hydrogel composition comprising:
(a) at least one antimicrobial silver salt;
(b) at least one viscosity-enhancing agent chosen from natural clay and synthetic clay; and
(c) at least one electrolyte.

2. The composition of claim 1, wherein the at least one antimicrobial silver salt is silver lactate.

3. The composition of claim 1, wherein the at least one antimicrobial silver salt is present in the composition in an amount ranging from about 0.01% to about 10% by weight relative to the total weight of the composition.

4. The composition of claim 3, wherein the at least one antimicrobial silver salt is present in the composition in an amount ranging from about 0.01% to about 5% by weight relative to the total weight of the composition.

5. The composition of claim 1, wherein the at least one antimicrobial silver salt is chosen from silver nitrate, silver acetate, silver citrate, silver picrate, and silver chloride.

6. The composition of claim 1, wherein the at least one viscosity-enhancing agent is a synthetic clay.

7. The composition of claim 1, wherein the at least one viscosity-enhancing agent further comprises at least one organic modifier.

8. The composition of claim 7, wherein the at least one organic modifier is chosen from hydroxypropyl methyl cellulose, guar hydroxypropyl trimonium chloride, carbomer, xanthan gum, polyethylene glycol block polymers, and polyvinylpyrrolidone.

9. The composition of claim 1, wherein the at least one viscosity-enhancing agent is a synthetic sodium lithium magnesium silicate.

10. The composition of claim 1, wherein the at least one viscosity-enhancing agent is present in the composition in an amount ranging from about 0.1 to about 10% by weight relative to the total weight of the composition.

11. The composition of claim 1, wherein the at least one viscosity-enhancing agent is chosen from magnesium aluminum silicates, smectite clays, allophone, kaolinite, nacarite, halloysites, sodium montmorillonite, calcium montmorillonite, sauconite, vermiculite, nontronite, saponite, hectorite, bentonite, attapulgite, sepiolite, palygorskite, and mixtures thereof.

12. The composition of claim 1, wherein the at least one electrolyte is present in the composition in an amount ranging from about 0.01 to about 10% by weight relative to the total weight of the composition.

13. The composition of claim 1, wherein the at least one electrolyte is chosen from sodium chloride USP, citric acid USP, and hydrochloric acid NF.

14. The composition of claim 13, wherein the at least one electrolyte is sodium chloride USP.

15. The composition of claim 13, wherein the at least one electrolyte is citric acid USP.

16. The composition of claim 13, wherein the at least one electrolyte is hydrochloric acid NF.

17. The composition of claim 1, wherein the at least one electrolyte is chosen from alkali metal salts and alkaline earth metal salts.

18. A method of topically disinfecting a substrate comprising applying to the substrate an effective amount of a composition comprising at least one antimicrobial silver salt; at least one viscosity-enhancing agent chosen from natural clay and synthetic clay; and at least one electrolyte.

19. The method of claim 18, wherein the substrate is skin.

20. The method of claim 18, wherein the substrate is a wound.

21. A method of treating a topical infection comprising applying to a patient in need thereof an effective amount of a disinfectant composition comprising at least one antimicrobial silver salt; at least one viscosity-enhancing agent chosen from natural clay and synthetic clay; and at least one electrolyte to the infected area and/or the surrounding infected area.

22. A method of treating a heavily contaminated or infected wound comprising applying to a patient in need thereof an effective amount of a composition comprising at least one antimicrobial silver salt; at least one viscosity-enhancing agent chosen from natural clay and synthetic clay; and at least one electrolyte to the contaminated or infected wound and/or the surrounding contaminated or infected area.

23. A method of disinfecting an intact skin site prior to a surgical or invasive procedure comprising applying to a patient in need thereof an effective amount of a composition comprising at least one antimicrobial silver salt; at least one viscosity-enhancing agent chosen from natural clay and synthetic clay; and at least one electrolyte.

24. A method for making an antimicrobial hydrogel composition comprising:
(a) combining at least one viscosity-enhancing agent chosen from natural clay and synthetic clay with water;
(b) combining at least one antimicrobial silver salt with water;
(c) combining the silver salt solution from (b) with the viscosity-enhancing solution from (a) to form a thickened solution; and
(d) combining at least one electrolyte with the thickened solution to form the antimicrobial hydrogel composition.
